(19) United States
(12) Patent Application Publication
(10) Pub. No.: US 2005/0272027 A1
(43) Pub. Date: Dec. 8, 2005

(54) Real-time clinical diagnostic systems for fluorescent spectrum analysis of tissue cells and methods thereof

(75) Inventors: Yi-Ping Cheng, Hsinchu City (TW); Chih-Chung Wu, Hsinchu City (TW); Sou-Lin Fong, Taichung City (TW); Kuo-Feng Hung, Caotun Township (TW); Jen-Hao Chang, Pusin Township (TW); Yuh-Ping Tong, Hsinchu City (TW); Mei-Fang Su, Hsinchu (TW); Su-Jan Lee, Taipei City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Mediatek Incorporation

(21) Appl. No.: 11/107,258

(22) Filed: Apr. 15, 2005

(30) Foreign Application Priority Data
Jun. 7, 2004 (TW) 93116311

(51) Int. Cl. C12Q 1/00; G06F 19/00; G01N 33/48; G01N 33/50; C12M 1/34

(52) U.S. Cl. 435/4; 702/19; 435/287.1

(57) ABSTRACT

Real-time clinical diagnostic expert systems for fluorescent spectrum analysis of tissue cells and methods thereof. An exemplary system includes a set of optical fibers, wherein the first optical fiber introduces an incident light to an subject epidermal tissue, and the second optical fiber receives an auto-fluorescent signal, a set of monochromators, wherein the first monochromator produces the incident light, and the second monochromator produces the auto-fluorescent signal from the second optical fiber, a light detector for detecting the auto-fluorescent signal from the second monochromator, a signal processing unit for plotting a spectrum of the auto-fluorescent signal, and a spectrum analyzing unit comprising a database for analyzing the spectrum with the database to obtain a probability of disease for the subject epidermal tissue.

### BACKGROUND

The invention relates to real-time diagnostic systems, and more particularly, to real-time clinical diagnostic expert systems for fluorescent spectrum analysis of tissue cells.

Cancer diagnosis requires a biopsy to detect cellular changes. The results of conventional biopsy typically requires longer than one week, which is both emotionally trying and potentially dangerous for a patient. Among various cancers, oral cavity cancer and skin cancer can be detected at the earliest stage and are mostly curable. The cure rate for oral cavity cancer in its early stages is relatively high at about 70˜80%, with a 5-year survival rate. This decreases to less than 50%, however, for late stage patients, or even 20% for patients having distant metastasis. Most skin cancer can be treated with simple surgery or radiotherapy if detected early. Skin cancer, including basal cell epithelioma, squamous cell carcinoma, and malignant melanoma, is almost benign. It is found that basal cell epithelioma rarely metastasizes, about 2% of squamous cell carcinoma has metastasized when the final diagnosis is made, especially when occurring in ears, cheeks, temples, and mucosa. Malignant melanoma typically metastasizes in the early stage. The mortality of skin cancer depends on the clinical stage and the occurrence of metastasis when treatment begins. Basal cell epithelioma has a recurrence of only 2%, squamous cell carcinoma about 92% with a 5-year survival rate, and the mortality of malignant melanoma depends on the diagnostic stage. In Taiwan, oral cavity cancer mostly occurs in male and skin cancer mostly in female according to a statistical analysis of Taiwan Department of Health records. In addition, the mortality rate from oral cavity cancer has be increasing.

In the United States, about 30,000 new cases of oral cavity cancer were diagnosed in 2001, and the death was about 7800 according to the report of the American Cancer Society in 2001. As for skin cancer, new cases were about 56,000 with almost 10,000 deaths. In particular, new cases of the life-threatening skin cancer, melanoma, has increased greatly in 20 years.

With the increasing danger of oral cavity cancer and skin cancer, development of a real-time, non-invasive clinical detection system for epidermal tissues is desirable.

Attempts to detect the auto-fluorescence of epidermal tissues mainly utilize a single characteristic for recognition. For example, U.S. Pat. No. 6,405,070, U.S. Pat. No. 6,405,074, WO 99/65394, and WO. 01/69199 to Bhaskar Banerjee disclose methods for the recognition of cancer cells and normal cells by fluorescent intensity at some specific wave lengths. U.S. Pat. No. 6,174,291 and WO 99/45838 to Brian T. McMahon disclose a complicated process for calculating characteristic values at several designated wavelengths to determine normal tissue, hyperplastic tissue, adenomatous tissue, or adenocarcinomas. This process can be classified as procedural representation schemes such as “If . . . Then . . . ”, and forward inference in the expert system classification. In addition, U.S. Pat. No. 6,289,236 to Frank Koenig discloses a method for distinguishing inflamed tissues from cancerous tissues by fluorescent intensity at a specific wavelength. These systems have many problems, thus, a need for a real-time, non-invasive clinical diagnostic system for epidermal cells is desirable.

### SUMMARY

Real-time, non-invasive clinical diagnostic expert systems for fluorescent spectrum analysis of tissue cells are provided. The fluorescent spectrum analysis of tissue cells may detect cellular changes, such as pathological changes, bacterial infection, hyperplasia, cancerous formation, or tumor growth. An exemplary embodiment of an expert system comprises a set of optical fibers where the first optical fiber introduces an incident light to a subject epidermal tissue and the second optical fiber receives an auto-fluorescent signal, a set of monochromators where the first monochromator produces the incident light and the second monochromator produces the auto-fluorescent signal from the second optical fiber, a light detector for detecting the auto-fluorescent signal from the second monochromator, a signal processing unit for plotting a spectrum of the auto-fluorescent signal, and a spectrum analyzing unit comprising a database for analyzing the spectrum with the database to obtain a disease probability for the subject epidermal tissue.

Methods for real-time, non-invasive clinical diagnosis for fluorescent spectrum analysis of tissue cells are also provided. An exemplary embodiment of a method comprises introducing an incident light produced by a first monochromator to a subject epidermal tissue through a first optical fiber, receiving an auto-fluorescent signal produced by the subject epidermal tissue through a second optical fiber to a second monochromator, detecting the auto-fluorescent signal from the second monochromator by a light detector, plotting a spectrum of the auto-fluorescent signal by a signal processing unit, and analyzing the spectrum of the auto-fluorescent signal with a database in a spectrum analyzing unit to obtain a disease probability for the subject epidermal tissue.

The analysis provides a comprehensive comparison for a plurality of spectrum characteristics such as fluorescent intensity at some specific wavelengths, spectral area at a specific range of wavelength, rising slope of a specific peak. A weight table can be created by these characteristics. The weight is obtained by classification and analysis of the collected tissues. The weight assumption is applied to differentiate diseases with similar characteristics. The calculation of the analysis is similar to frame-based knowledge representation and probability-based assumption in the classification of the expert system, which is different from the conventional methods.

### BRIEF DESCRIPTION OF THE DRAWINGS

Real-time diagnostic system for fluorescent spectrum analysis of tissue cells and methods thereof can be more fully understood and further advantages become apparent when reference is made to the following description and the accompanying drawings in which:

FIG. 1A is a diagram showing an embodiment of a real-time, non-invasive clinical diagnosis expert system for fluorescent spectrum analysis of tissue cells.

FIG. 1B is a photograph showing the embodiment of the real-time, non-invasive clinical diagnosis expert system for fluorescent spectrum analysis of tissue cells of FIG. 1A.

FIG. 2 illustrates the calculation of the embodiment of the real-time, non-invasive clinical diagnosis expert system for fluorescent spectrum analysis of tissue cells of FIG. 1A.

FIG. 3 is a diagram showing the construction of the weight table of the embodiment of the real-time, non-invasive clinical diagnosis expert system for fluorescent spectrum analysis of tissue cells of FIG. 1A.

FIG. 4 is a diagram showing the automatic correction of the embodiment of the real-time, non-invasive clinical diagnosis expert system for fluorescent spectrum analysis of tissue cells of FIG. 1A.

FIG. 5 is a photograph showing the optical fiber of the embodiment of the real-time, non-invasive clinical diagnosis expert system for fluorescent spectrum analysis of tissue cells of FIG. 1A.

FIG. 6 is a photograph showing the measurement of a human epidermal tissue by an optical fiber in Example 1.

FIG. 7A˜7C illustrate fluorescent spectra of different normal volunteers. FIG. 7A is the spectrum of volunteer No. 1; FIG. 7B is of No. 2; and FIG. 7C is of No. 3.

FIG. 8A˜8D illustrate fluorescent spectra at different amino acid concentrations. FIG. 8A is the spectrum of tyrosine; FIG. 8B˜8D is of tyrosine and phenylalanine. FIG. 8B: 300 nm of incident light, 310˜580 nm of scanning range; FIG. 8C: 300 nm of incident light, 330˜620 nm of scanning range; FIG. 8D: 320 nm of incident light, 325˜620 nm of scanning range.

FIG. 9A˜9B illustrates spectra of different culture cells. FIG. 9A: 280 nm of incident light, 290˜540 nm of scanning range; FIG. 9B: 420 nm of incident light, 440˜820 nm of scanning range.

### DETAILED DESCRIPTION

Real-time clinical diagnostic expert systems for fluorescent spectrum analysis of tissue cells and methods thereof are provided.

An embodiment of a real-time clinical diagnostic expert system for fluorescent spectrum analysis of tissue cells comprises a fluorescent spectrum database for epidermal tissues. In clinical application, an embodiment of the real-time clinical diagnostic expert system may be used prior to biopsy. When epidermal tissue is determined to be cancerous, the result can then be confirmed by biopsy. An embodiment of the expert system is mainly applicable to oral cavity cancer and skin cancer since fluorescent spectra of the epidermal tissues from these cancers can be obtained easily.

Practical examples are given in the following.

1. Establishment of an Embodiment of a Real-Time Clinical Diagnostic Expert System for Fluorescent Spectrum Analysis of Tissue Cells.

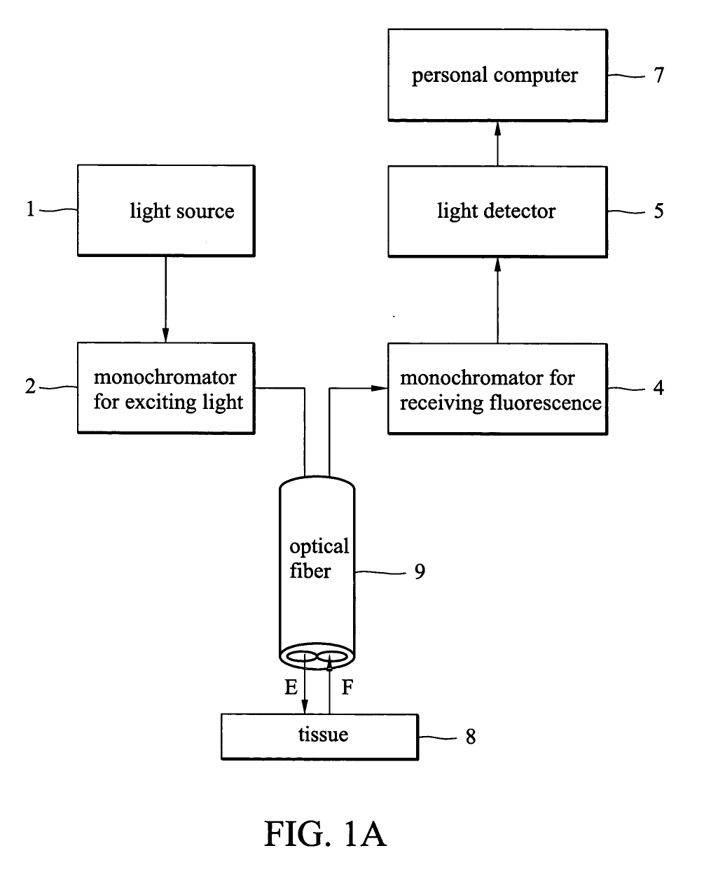

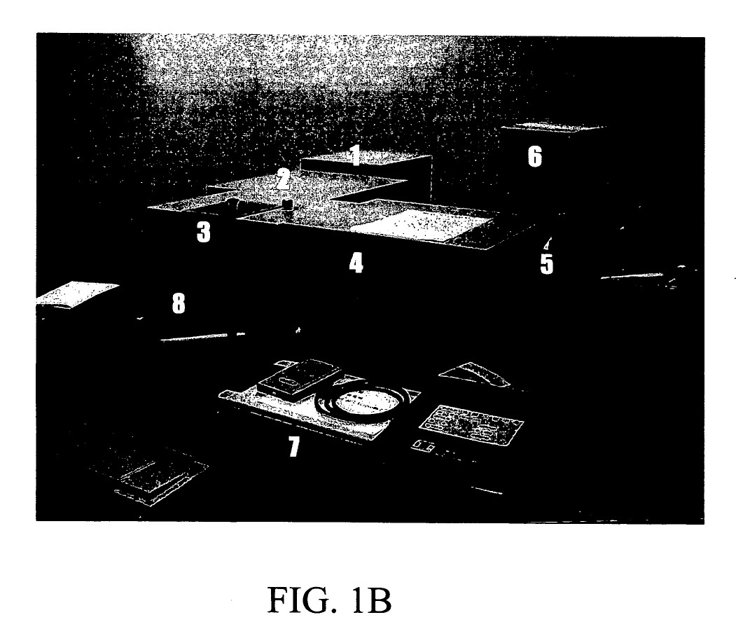

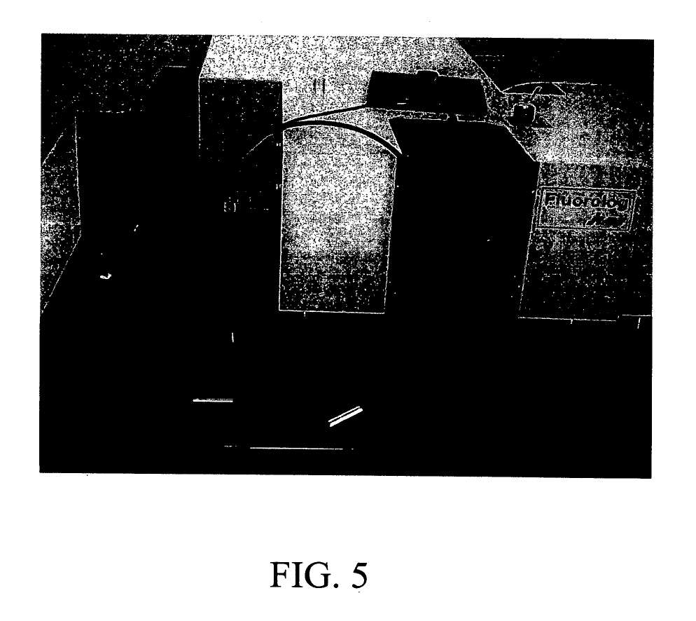

An embodiment of a real-time clinical diagnostic expert system for fluorescent spectrum analysis of tissue cells are illustrated in FIGS. 1A and 1B. The expert system comprises a light source 1 for producing an incident light, a set of monochromators, one for incident light E at a specific wavelength (the first monochromator 2), the other for receiving fluorescence F at a specific wavelength (the second monochromator 4), a sample platform 3, for example, the sample 8 can be placed thereon as shown in FIG. 1B, or a set of optical fibers for introducing the incident light to the epidermal tissue or receiving the auto-fluorescence produced by the epidermal tissue as shown in FIG. 5, a light detector 5 for receiving the auto-fluorescent signal, and a controlling computer 6 as well as a data-processing computer 7. The pathological changes in the epidermal tissue can be determined by the spectral characteristics of the auto-fluorescence.

2. Design of the Calculation of an Embodiment of the Clinical Diagnosis Expert System

The determination of the fluorescence spectrum combines several representations and inference. The calculation was designed in the combination of logic knowledge representation and interference. In addition, probability was applied in the calculation according to the experience of medical expert systems of the inventors. Moreover, the calculation was based on a plurality of spectral characteristics.

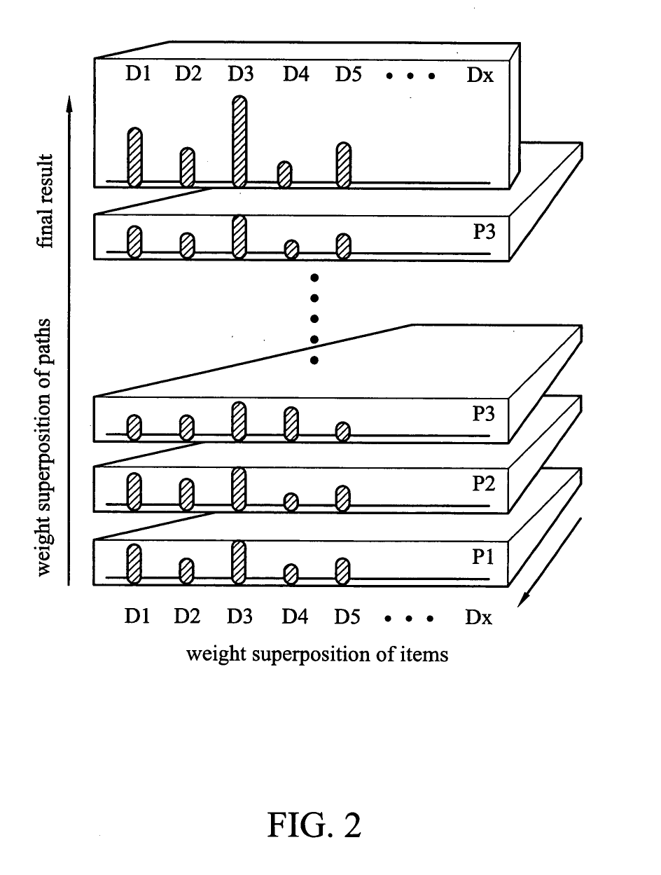

FIG. 2 is a diagram showing the calculation of an embodiment of the expert system. In FIG. 2, P1, P2, . . . , and P7 indicate properties, i.e. the spectral characteristics, and 1, 2, . . . , and 7 represent serial numbers, for example, P1 represents area ratio, P2 represents intensity ratio, P3 represents slope, . . . , and P7 represents area ratio 2. The calculation may extend the amount of the properties and those skilled in the art may define any desired properties. D1, D2, D3, . . . , Dₓ indicate diseases or symptoms which are not limited and may be defined by the user if required.

The primary basis of the calculation is that spectra of different diseases have more than one property and disease properties may overlap. If two conditions, for example, normal and cancerous tissues, are compared, one property difference in the spectrum is enough to distinguish between them. Only one property difference, however, is not enough to differentiate between more than three kinds of tissues. The calculation is based on the disease probability corresponding to spectral properties in a core database. A higher probability of a disease corresponding to a spectral property indicates that the possibility of the disease is higher. Since probability is a statistical result, a large sample population may simulate clinical diagnosis made by the physicians. The probability of a certain disease can be calculated in corresponding to a spectral property of a sample tissue.

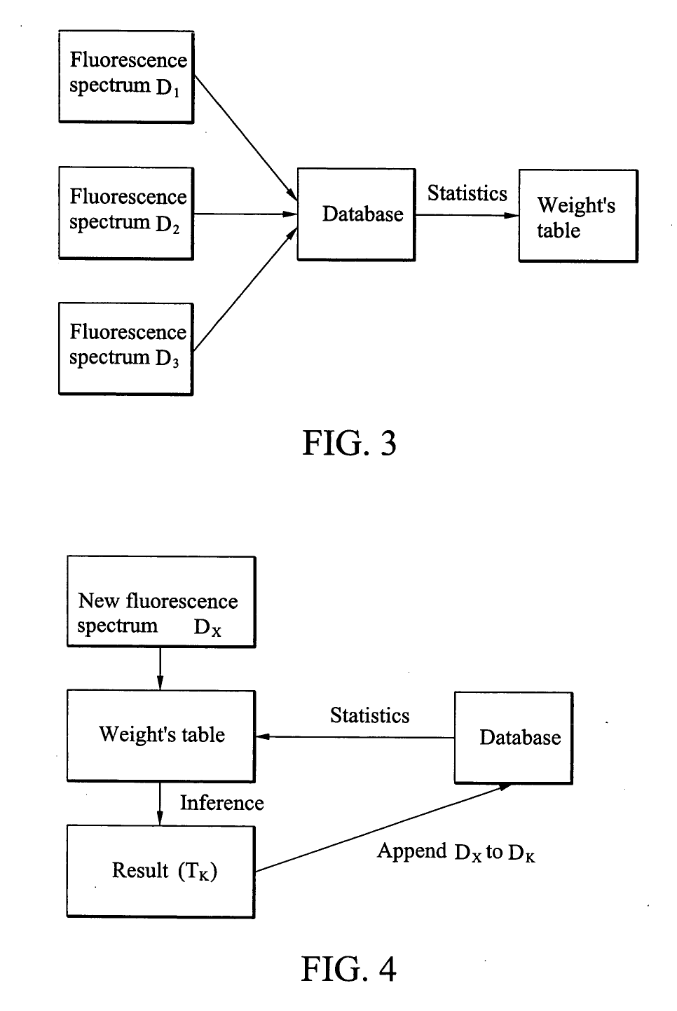

Accordingly, the calculation is based on the probability of diseases corresponding to the spectral properties and the assumption of these probabilities may create a weight table as shown in FIG. 3. In FIG. 3, the database contains property table of many diseases or symptoms, and the core weight table may be calculated accordingly. The calculation recited below.

The database of the calculation contains several tables which represent different definitions. Each disease, defined as D, indicates a class containing an independent table. Therefore, the sample population of the disease database is:
D={D₁, D₂, D₃, . . . , Dₖ}, where k∈N (1)
wherein D₁, D₂, D₃, . . . , Dₖ indicate independent tables respectively, where 1, 2, 3, . . . , k represent the types of diseases, individually, Disease 1, Disease 2, Disease 3, . . . , Disease k, which is defined by the user.

An assumption of spectral properties is in each disease table and can be defined as Dₓ, and the sample population can be represented as:
Dₖ={bᵢⱼ}, wherein i,j∈N (2)
wherein b represents Boolean value, indicating sample j of disease Dₖ corresponds to a Boolean value of property i.

The type or title of disease or symptom is defined prior to creation of the database. The samples used in the database are known as belonging to a type of disease. For example, for the determination of the spectra of basal cell epithelioma, squamous cell carcinoma, malignant melanoma, psoriasis, and nevus, the databases of the spectra are numbered as D₁, D₂, D₃, D₄, and D₅, respectively. The sample can be created in its own database Dₖ in accordance with the defined types or titles of the diseases and symptoms.

In each database Dₖ, the statistical probability S of each spectral property can be obtained by biostatistic method as shown below:
Sₙₖ=P(Pₙ|Dₖ), where n,k∈N (3)
wherein Sₙₖ represents the probability P(|) of the spectral property n in disease k. After the statistical probability is established in each spectral property database of each diseased tissue, the core weight table (W) can be created.
   W={Sₙₖ}, where n,k∈N (4)

Formula (4) is the representation of the sample size in the weight table W, composed of Sₙₖ.

The database and weight table can be established accordingly.

The inference of the expert system is illustrated below. When a spectrum of a new sample tissue is produces, a Boolean array corresponding to each spectral property, represented as Dₓ, is created. The array is:

Dₓ={bᵢ}, where i∈N (5)

wherein Dₓ represents a unknown disease, bᵢ represents the Boolean value of spectral property i in the unknown tissue.

When Dₓ, is created, the inference can be made based on the weight table W. The inference formula is:

$${T}_{k} = {\sum }_{i = 1}^{n} ( {S}_{i , k} \vert {b}_{i} = true ) \qquad ( 6 )$$

The inference is determined by Tₖ, representing the sum of the probability of disease Dₖ corresponding to Dₓ. The higher the sum of the probability of a certain disease, the higher possibility the disease has. Therefore, an inference can be made by using this formula.

The calculation further comprises an auto-modification of the weight table, as shown in FIG. 4. When the inference result Tₖ is known, Dₓ can be appended to database Dₖ, and Sₙₖ of weight table W can be automatically modified.

The spectral property of an embodiment of the clinical diagnosis expert system for fluorescent spectrum analysis of tissue cells is not limited and can be any user-defined properties. The diseases or symptoms are not limited and can be flexibly defined and combined with any spectral properties. The calculation is based on probability, and the establishment of the sample population and the probability of the diseases corresponding to a defined spectral property create the core database for the calculation. The assumption of the probability is correlated to the possibility of a certain disease. In addition, the calculation provides probability of other diseases as a reference for diagnosis. The probability information is different from the positive and negative determination method in the conventional methods.

Generally, the diagnostic method for auto-fluorescent spectrum analysis of tissue cells usually utilizes ultraviolet light at 280 nm to obtain fluorescent spectrum from tissue cells. It was reported that auto-fluorescence is obtained from proteins such as elastin, amino acids such as tryptophan, tyrosine, or phenylalanine, purines such as adenine or guanine, pyrimidines, nucleic acids such as adenosine, guanosine, DNA or RNA, which absorb ultraviolet at 280 nm and produce peaks at 340˜390 nm. Still no study focuses on the auto-fluorescent spectral properties of a simple material such as different amino acids. This property relates to the stages or conditions of a disease, for example, the fluorescent spectra of cancerous and normal tissues are different in the amount of amino acids produced. Amino acids at different concentration are applied in the establishment of the spectral database and for the verification of the calculation. Database Dₖ is not limited in disease titles, amino acid in different concentration or with different types are also applicable.

Practical examples of the invention uses pathologic cell cultures in state of patient cells. The fluorescent spectra of cells obtained from a culture or a patient should be similar since the cellular components are the same.

For safety considerations, the incident light of an embodiment of the expert system can be modified by the wavelength, for example, the wave range can be from infrared to ultraviolet, preferably green light.

Practical examples are described herein.

### EXAMPLES

### Example 1

### Measurement of the Fluorescent Spectrum from Human Epidermal Tissue

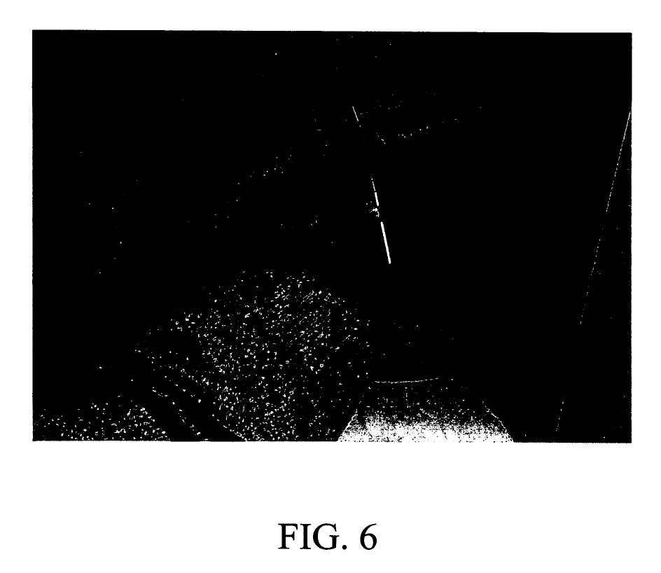

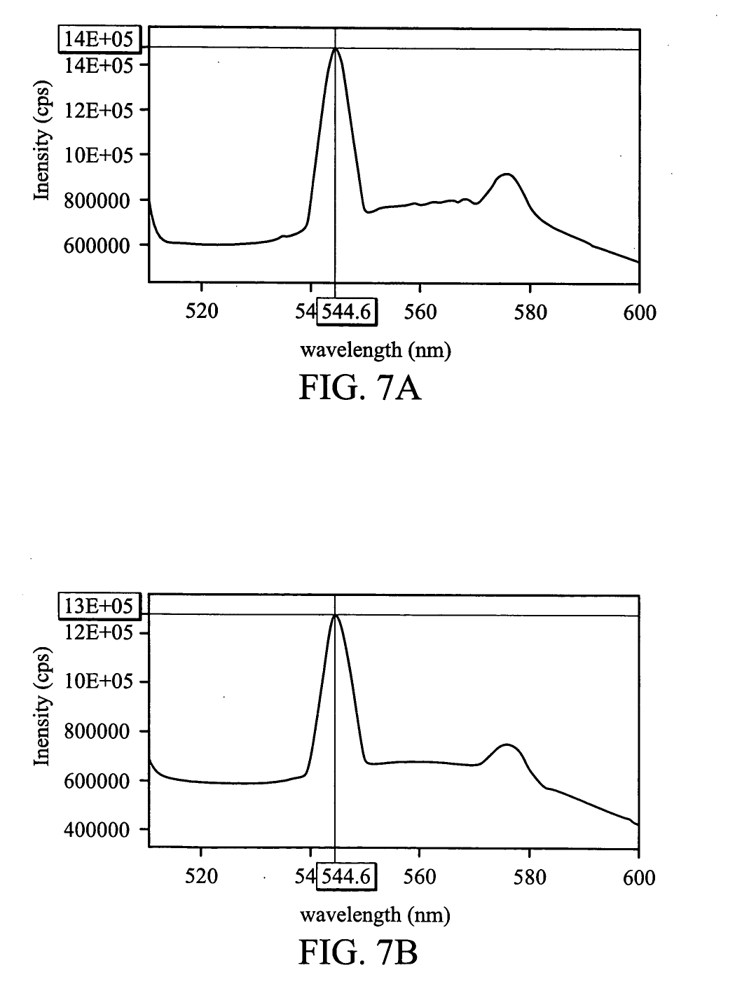

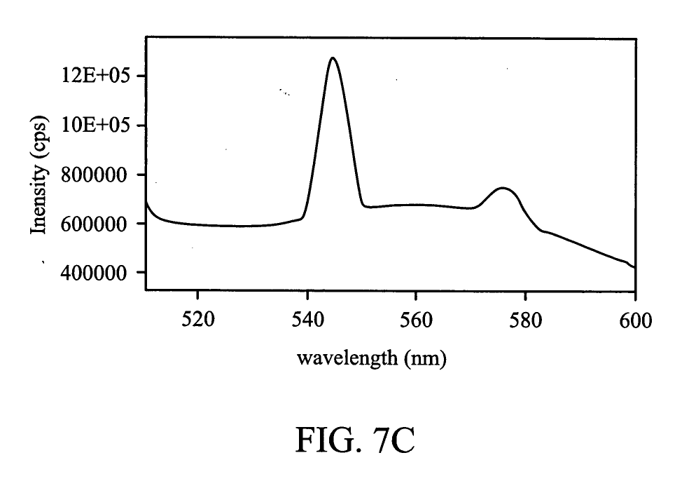

The measurement was made by the device as shown in FIG. 1B, and the sample platform 3 was modified as a set of optical fibers as shown in FIG. 5. The auto-fluorescent spectrum was obtained from the epidermal tissue of a normal subject with the method as shown in FIG. 6. The light source is green light at 500 nm, the scanning spectrum is from 510 nm to 600 nm. The results of three normal subjects are shown in FIG. 7A˜7C. It is found that a peak is located at 544.6 nm, indicating auto-fluorescent spectrum can be obtained from epidermal tissues by green light as the incident light, which is not recited in any records.

### Example 2

### Measurement of Fluorescent Spectra for Amino Acids at Different Concentration

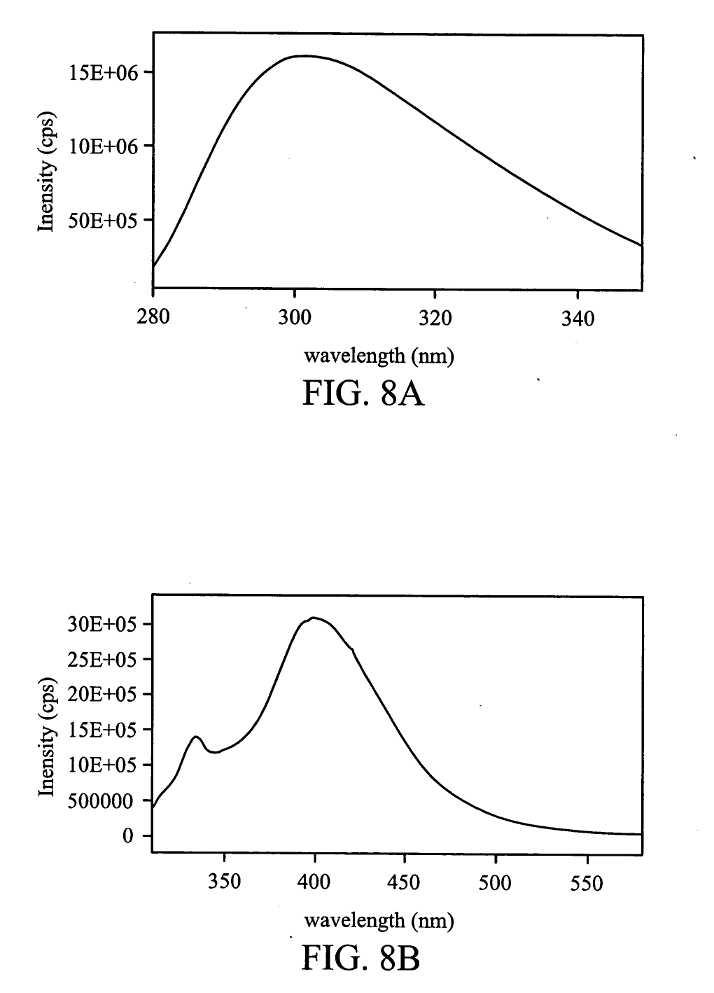

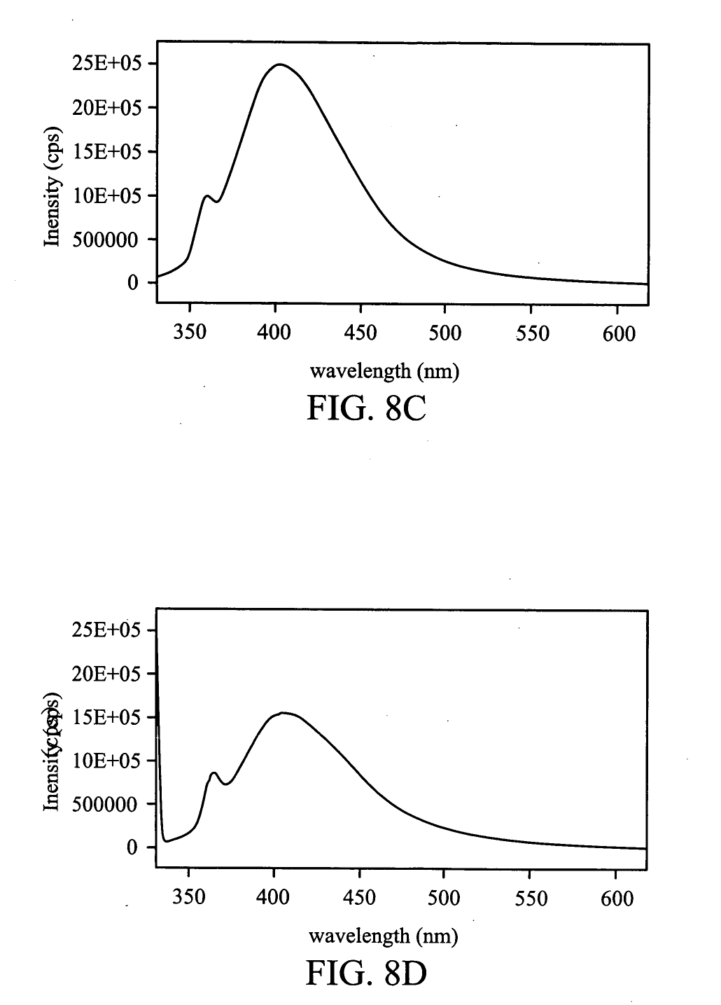

The measurement was made by the device as shown in FIG. 1B, and the spectra of amino acids at different concentration are shown in FIG. 8A˜8D. FIG. 8A shows the fluorescent spectrum of tyrosine at 0.05 mg/ml by a light source at a wavelength of 300 nm. FIG. 8B˜8D shows the spectra of phenylalanine at 0.005 mg/ml and tyrosine at 0.05 mg/ml. The light source of FIG. 8A is at a wavelength of 300 nm in a scanning range of 310 nm˜580 nm; it of FIG. 8C is at 320 nm in a scanning range of 330 nm˜620 nm; it of FIG. 8D is at 320 nm in a scanning range of 325 nm˜620 nm.

The results indicate that different wave peaks of the fluorescent spectra represent the mixture at different concentration. This is the basic rule for the establishment of spectra database of cellular components.

### Example 3

### Measurement of Fluorescent Spectra of Different Culture Cells

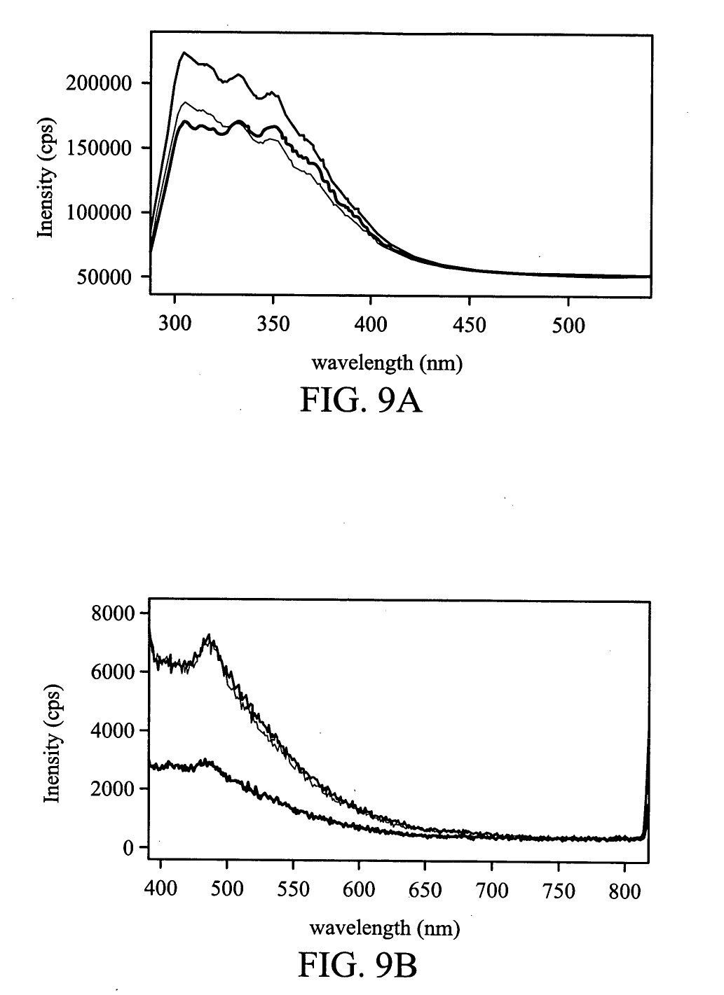

Measurement was made for different culture cells by the device as shown in FIG. 1B. The spectral results of hepatoma cells and melanoma cells are shown in FIG. 9A˜9B. The incident light is ultraviolet at 280 nm.

In FIG. 9A, the curve lines are PBS, PBS+hepatoma cells, and PBS+melanoma cells from the top to the bottom, the incident light is ultraviolet at 280 nm, and the scanning range is from 290 nm to 540 nm. In FIG. 9B, the curve represents PBS, PBS+melanoma cells, and PBS+hepatoma cells, the incident light is violet light at 420 nm, and the scanning range is from 440 nm to 820 nm. PBS indicates the solution in the culture. The results show that cancer cells can be differentiated by different scanning ranges.

Recently, increasing attempts focus on optical measurement for cancer analysis. The principles which can be applied include scattering, laser response, wavelength changes, auto-fluorescence, dye fluorescence, and so on. From the disclosed experimental data, auto-fluorescent properties as well as other optical properties may be useful for cancer cell analysis in the application of the disclosed calculation.

While the invention has been described by way of example and in terms of preferred embodiment, it is to be understood that the invention is not limited thereto

1. A real-time clinical diagnosis expert system for fluorescent spectrum analysis of tissue cells, comprising:
a set of optical fibers comprising a first optical fiber for introducing an incident light to a subject epidermal tissue, and a second optical fiber for receiving an auto-fluorescent signal produced by the subject epidermal tissue;
a set of monochromators comprising a first monochromator for producing the incident light and a second monochromator for receiving the auto-fluorescent signal received by the second optical fiber;
a light detector for detecting the auto-fluorescent signal received by the second monochromator;
a signal processing unit for plotting a spectrum of the auto-fluorescent signal, and
a spectrum analyzing unit comprising a database for analyzing the spectrum with the database to obtain a disease probability for the subject epidermal tissue.

2. The system as claimed in claim 1, wherein the signal processing unit plots the auto-fluorescent signal with a weight table (W), and the weight table (W) is obtained from a serial process comprising:
combining a plurality of diseases (D) and a plurality of signals to obtain an assumption (Dₖ),
transferring the assumption to a spectral probability (S), and
inferring the spectral probability (S) a weight table (W);
wherein
the plurality of diseases (D) are as formula (1):
D={D₁, D₂, D₃, . . . , Dₖ} (1)
wherein D₁, D₂, D₃, . . . , Dₖ indicate the type of diseases, k is a natural number;
the assumption of the plurality of signal in each disease is as formula (2):
Dₖ={bᵢⱼ} (2)
wherein b indicates Boolean values, representing a Boolean value of signal i corresponding to sample j of disease Dₖ, and i and j are natural numbers;
the signal probability (S) is as formula (3):
Sₙₖ=P(Pₙ|Dₖ) (3)
wherein Sₙₖ represents a statistic probability P(|) of signal n of disease k, and n, k are natural numbers;
the weight table (W) is as formula (4):
W={Sₙₖ} (4)
wherein n, k are natural numbers.

3. The system as claimed in claim 2, wherein the auto-fluorescent signal is as formula (5):
Dₓ={bᵢ} (5)
wherein Dₓ represents a disease of the subject epidermal tissue, bᵢ represents a Boolean value of signal I of the subject epidermal tissue, and i is a natural number.

4. The system as claimed in claim 3, wherein the spectrum analyzing unit analyzes the spectrum of the auto-fluorescent signal and the database by formula (6):
$${T}_{k} = {\sum }_{i = 1}^{n} ( {S}_{i , k} \vert {b}_{i} = true ) \qquad ( 6 )$$
wherein Tₖ represents a sum of the probability for Dₓ corresponding to Dₖ for inferring Dₓ to a defined disease, and the higher Tₖ is, the higher possibility of the defined disease.

5. The system as claimed in claim 4, further comprising an auto-modification of the weight table, the auto-modification of the weight table automatically appends a result of Dₖ to Sₙₖ.

6. The system as claimed in claim 1, wherein the incident light is green light.

7. The system as claimed in claim 1, wherein the spectrum property comprises a fluorescent intensity of a defined wavelength, an area of a defined wavelength range, or a slope of a defined wave peak.

8. The system as claimed in claim 1, wherein the disease comprises basal cell epithelioma, squamous cell carcinoma, malignant melanoma, psoriasis, or nevus.

9. A clinical diagnosis method for fluorescent spectrum analysis of tissue cell, comprising:
introducing an incident light produced by a first monochromator to a subject epidermal tissue through a first optical fiber;
receiving an auto-fluorescent signal produced by the subject epidermal tissue through a second optical fiber to a second monochromator;
detecting the auto-fluorescent signal from the second monchromator by a light detector;
plotting a spectrum of the auto-fluorescent signal by a signal processing unit; and
analyzing the spectrum of the auto-fluorescent signal with a database in a spectrum analyzing unit to obtain a disease probability for the subject epidermal tissue.

10. The method as claimed in claim 9, wherein the signal processing unit plots the auto-fluorescent signal with a weight table (W), and the weight table (W) is obtained from a serial process comprising:
combining a plurality of diseases (D) and a plurality of signals to obtain an assumption (Dk),
transferring the assumption to a spectral probability (S), and
inferring the spectral probability (S) a weight table (W);
wherein
the plurality of diseases (D) are as formula (1):
D={D₁, D₂, D₃, . . . , Dₖ} (1)
where D₁, D₂, D₃, . . . , Dₖ indicate the type of diseases, k is a natural number;
the assumption of the plurality of signal in each disease is as formula (2):
Dₖ={bᵢⱼ} (2)
wherein b indicates Boolean values, representing a Boolean value of signal i corresponding to sample j of disease Dₖ, and i and j are natural numbers;
the signal probability (S) is as formula (3):
Sₙₖ=P(Pₙ|Dₖ) (3)
wherein Sₙₖ represents a statistic probability P(|) of signal n of disease k, and n, k are natural numbers;
the weight table (W) is as formula (4):
W={Sₙₖ} (4)
wherein n, k are natural numbers.

11. The method as claimed in claim 10, wherein the auto-fluorescent signal is as formula (5):
Dₓ={bᵢ} (5)
wherein Dₓ represents a disease of the subject epidermal tissue, bᵢ represents a Boolean value of signal i of the subject epidermal tissue, and i is a natural number.

12. The method as claimed in claim 11, wherein the spectrum analyzing unit analyzes the spectrum of the auto-fluorescent signal and the database by formula (6):
$${T}_{k} = {\sum }_{i = 1}^{n} ( {S}_{i , k} \vert {b}_{i} = true ) \qquad ( 6 )$$
wherein Tₖ represents a sum of the probability for Dₓ corresponding to Dₖ for inferring Dₓ to a defined disease, and the higher Tₖ indicates a higher possibility of the defined disease.

13. The method as claimed in claim 12, further comprising a step of auto-modification of the weight's table, the auto-modification of the weight table automatically appends a result of Dₖ to Sₙₖ.

14. The method as claimed in claim 9, wherein the incident light is green light.

15. The method as claimed in claim 9, wherein the spectrum property comprises a fluorescent intensity of a defined wavelength, an area of a defined wavelength range, or a slope of a defined wave peak.

16. The method as claimed in claim 9, wherein the disease comprises basal cell epithelioma, squamous cell carcinoma, malignant melanoma, psoriasis, or nevus.
